(19) United States
(12) Patent Application Publication
(10) Pub. No.: US 2026/0355143 A1
(43) Pub. Date: Nov. 12, 2026

(54) THIMEROSOL FORMULATION FOR VIRAL INHIBITION

(71) Applicant: FIRST PERSON SOLUTIONS, LLC, Media, PA (US)

(72) Inventor: Joel ADAMS, Sydney NSW (AU)

(21) Appl. No.: 19/155,458

(22) PCT Filed: Feb. 12, 2024
(86) PCT No.: PCT/IB2024/051267

§ 371 Date: Aug. 11, 2025

Related U.S. Application Data
(60) Provisional application No. 63/444,730, filed on Feb. 10, 2023.

(51) Int. Cl. A61K 31/305; A61K 31/336; A61K 31/4375; A61K 31/4525; A61K 31/473; A61K 31/4745; A61K 31/575; A61K 31/7034; A61K 31/7048; A61K 36/59; A61K 36/67; A61P 31/14

(52) U.S. Cl. CPC A61K 31/305; A61K 31/336; A61K 31/4375; A61K 31/4525; A61K 31/473; A61K 31/4745; A61K 31/575; A61K 31/7034; A61K 31/7048; A61K 36/59; A61K 36/67; A61P 31/14

(57) ABSTRACT

The technology relates to the use of a formulation for inhibiting a coronavirus or to treat, prevent, or reduce the risk of infection by the coronavirus, or reduce a symptom of long-covid. The formulation comprises thimerosol, and optionally one or any combination of additional compounds selected from tinocordofolin, cordifolioside A, cordioside, berberine, syringin, tinocordioside, jateroine, jatrorrhizine, magnoflorine, 20-hydroxy ecdysone, and palmatine. The formulation may further comprise piperine.

### CROSS REFERENCE TO RELATED APPLICATION

This application claims priority to U.S. provisional application No. 63/444,730 filed 10 Feb. 2023, which is incorporated by reference in its entirety.

### TECHNICAL FIELD

The technology relates to the use of an orally administered formulation comprising thimerosal as an active to inhibit a coronavirus in a subject to prevent or treat an infection by the coronavirus.

### BACKGROUND

As of January 2023 more than 650,000,000 confirmed cases of COVID-19, including over 6,500,000 deaths, had been reported to the World Health Organization. COVID-19 has resulted in an unprecedented worldwide public health response and even with the administration of over 13 billion doses of vaccine globally COVID-19 has become endemic. Therapies for COVID-19 are limited to a small number of antiviral drugs or convalescent plasma that are indicated for use only in severe cases or for specific patient populations. In many patients therapeutic intervention is limited to management of symptoms.

Severe acute respiratory syndrome coronavirus 2 (SARS-CoV-2), is the causative agent of COVID-19. It remains unclear why COVID-19 patients experience a spectrum of clinical outcomes ranging from asymptomatic to severe disease. However, the salient features of COVID-19 pathogenesis and mortality are inflammation and cytokine release syndrome that can lead to acute respiratory distress syndrome (ARDS). Immune cell infiltration into the lung and ARDS have been described as defining features of severe disease in humans infected with the closely related betacoronaviruses, SARS-CoV and MERS-CoV.

Patients with chronic COVID-19 or ‘long covid’ experience a multitude of symptoms from several weeks to months after recovering from their acute illness and months after viral clearance. These symptoms include joint pain, muscle aches, fatigue, and ‘brain fog’. These symptoms can commonly resemble rheumatic diseases and autoimmune disorders, which involve inflammation, hyper- and/or auto-immunity and some such as chronic fatigue are associated with viral persistence after an acute infection with pathogens such as Epstein Barr and Cytomegalovirus.

The inventors have developed a composition comprising thiomersal as an active to treat and prevent infection by SARS-CoV-2 and all members of the Coronaviridae (Coronavirus) family. The composition is also useful for treating long covid.

### SUMMARY

In a first aspect, there is provided a formulation comprising thimerosol for inhibiting a corona virus in a subject.

The formulation may further comprise one or any combination of additional compounds selected from tinocordifolin, and one or more of cordifolioside A, cordioside, berberine, syringin, tinocordiside, jateorin, palmatine, jatrorrhizine, magnoflorine, and 20-hydroxy ecdysone.

The formulation may further comprise piperine.

In one embodiment the formulation further comprises fennel oil.

The formulation may further comprise one or more of a humectant, sweetener, preservative, flavouring agent, excipient, diluent, and carrier.

For example the humectant may be glycerine, propylene glycol, or both.

The sweetener may be sucralose.

The preservative may be sodium benzoate.

In one embodiment the flavouring agent may be an artificial pineapple flavour.

The formulation may also comprise sorbitol as an excipient.

In a preferred embodiment the formulation comprises, per 100 mL, about 0.05 mg thimerosol, about 200 mg of the additional compounds; about 2.5 mg piperine; about 19.23 mL 70% sorbitol; about 0.25 mL fennel oil; about 39.59 ml glycerine; about 4.81 mL propylene glycol; about 100 mg sucralose; about 300 mg sodium benzoate; and about 1.5 mL artificial pineapple flavour.

The additional compounds may be provided by a Tinospora cordifolia stem extract.

The piperine may be provided by a Piper nigrum seed extract

The subject may be a human.

The coronavirus may be coronavirus 229E, coronavirus NL63, coronavirus OC43, SARS-CoV, MERS-CoV, SARS-CoV-2, or variants thereof.

In one embodiment the coronavirus is SARS-CoV-2 or a variant thereof.

In a second aspect there is provided a method of inhibiting a coronavirus in a subject, the method comprising administering to the subject a therapeutically effective amount of the formulation of the first aspect.

In some embodiments the level of detectable coronavirus in the subject is reduced by at least 5%, at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9%.

In a third aspect there is provided a method of treating, preventing, or reducing the risk of a coronavirus infection in a subject with a coronavirus infection, the method comprising administering to the subject a therapeutically effective amount of the formulation of the first aspect.

In a fourth aspect there is provided a method for preventing antibody dependent enhanced (ADE) coronavirus infection in a subject, comprising administering to the subject a therapeutically effective amount of the formulation of the first aspect.

In a fifth aspect there is provided a method of reducing a symptom of a coronavirus infection, preferably a symptom of long-covid, in a subject comprising administering to the subject the formulation of the first aspect.

The level of detectable coronavirus is reduced over a period of 1, 2, 3, 4, 5, 6, 7, 8 or more days after administration of the formulation.

The formulation is preferably administered orally, preferably sub-lingually.

In some embodiments the formulation is administered over a period of 1, 2, 3, 4, 5, 6, 7, 8, 9, 10, 11, 12, 13, 14 or more days.

In one embodiment a dose of the formulation may be administered at least hourly on the first day, for example a dose of the formulation may be administered about every 15 minutes for the first hour and then every hour during waking hours.

On the second and third days of treatment a dose of the formulation may be administered at least four times a day, preferably at least six times a day.

On the fourth and subsequent days of treatment a dose of the formulation is administered at least three or four times a day, preferably at least six times a day, for example on the fourth to seventh days of treatment a dose of the formulation is administered four times a day.

After the seventh day of treatment a dose of the formulation may be administered three times a day.

In one embodiment a dose of the formulation may be administered three or four times a day

The subject is preferably a human.

The coronavirus may be coronavirus 229E, coronavirus NL63, coronavirus OC43, SARS-CoV, MERS-CoV or SARS-CoV-2.

In one embodiment the coronavirus is SARS-CoV-2 or a SARS-CoV-2 variant.

The symptom of long-covid may be persistent fatigue or inflammation.

The reduction in persistent fatigue may be characterized by a reduction in a FSSQ (Fatigue Severity Scale Questionnaire) score.

The reduction in inflammation may characterized by a reduction in IFN-γ and/or CCL4 (MIP-1β) levels.

The reduction of a symptom may be characterized by a decreased post-acute sequelae score of COVID-19 (PASC) Score.

In a sixth aspect there is provided use of thimerosol for the manufacture of a medicament for the prevention or treatment of a coronavirus infection, or to reduce a symptom of a coronavirus infection.

In an seventh aspect there is provided use of thimerosol for the manufacture of a medicament for reducing the risk of acquiring a coronavirus infection.

In a eighth aspect there is provided use of thimerosol for the preparation of a medicament for preventing antibody dependent enhanced (ADE) coronavirus infection in a subject.

### DEFINITIONS

Throughout this specification, unless the context clearly requires otherwise, the word ‘comprise’, or variations such as ‘comprises’ or ‘comprising’, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Throughout this specification, the term ‘consisting essentially of’ means the inclusion of the stated element(s), integer(s) or step(s), but other element(s), integer(s) or step(s) that do not materially alter or contribute to the working of the invention may also be included.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (i.e., at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

The terms ‘treating’, and ‘treatment’ are used herein to refer to curative therapy, prophylactic therapy, palliative therapy and preventative therapy. Thus, in the context of the present disclosure the term ‘treating’ encompasses curing, ameliorating or tempering the severity of a coronavirus infection or one or more of its associated symptoms.

The terms ‘effective amount’ or ‘therapeutically effective amount’ refer to an amount of the formulation disclosed herein sufficient to produce a desired therapeutic or pharmacological effect in the subject being treated. The terms are synonymous and are intended to qualify the amount the formulation that will achieve the goal of improvement in disease severity while preferably avoiding or minimizing adverse side effects, including side effects typically associated with other therapies. Those skilled in the art can determine an effective dose using information and routine methods known in the art.

More specifically, the terms ‘effective amount’ or ‘therapeutically effective level’ and similar terms means an amount of a formulation that (i) treats the particular disease, condition, or disorder, (ii) attenuates, ameliorates, or eliminates one or more symptoms of the particular disease, condition, or disorder, (iii) prevents or delays the onset of one or more symptoms of the particular disease, condition, or disorder described herein, (iv) prevents or delays progression of the particular disease, condition or disorder, or (v) reverses damage caused prior to treatment to some extent. The reversal does not have to be absolute. The amount administered may be higher than what is required within the body to achieve the therapeutic effect, but takes in to account the absorption, distribution, metabolism and excretion (ADME) profile of the formulation disclosed herein, the route of administration and the administration frequency.

In an alternative embodiment, the severity of symptoms associated with a coronavirus infection can be reduced by administering a therapeutically effective amount of the formulation disclosed herein to the subject. Reducing symptom severity means that one or more symptoms are attenuated, ameliorated, or eliminated, or the onset of one or more symptoms is prevented or delayed.

A ‘pharmaceutical carrier, diluent or excipient’ includes, but is not limited to, water or any physiological buffered (i.e., about pH 7.0 to 7.4) medium comprising a suitable water soluble organic carrier, conventional solvents, dispersion media, antibacterial and antifungal agents, isotonic and absorption delaying agents. Suitable water soluble organic carriers include, but are not limited to, saline, dextrose, corn oil, dimethylsulfoxide, and gelatin capsules.

As used herein the terms ‘long COVID’, ‘long covid’ and ‘post COVID-19 condition’ are used interchangeably to refer to the continuation or development of symptoms 3 months after an initial SARS-CoV-2 infection, with these symptoms lasting for at least 2 months with no other explanation.

While common symptoms of long COVID can include fatigue, joint pain, muscle aches, shortness of breath and cognitive dysfunction (‘brain fog’), over 200 different symptoms have been reported that can have an impact on patents with long COVID.

‘Subject’ includes any human or non-human mammal. Thus, in addition to being useful for human treatment, the compounds of the present invention may also be useful for veterinary treatment of mammals, including companion animals and farm animals, such as, but not limited to dogs, cats, horses, cows, sheep, camels, chickens and pigs. In preferred embodiments the subject is a human.

In the context of this specification the term ‘administering’ and variations of that term including ‘administer’ and ‘administration’, includes contacting, applying, delivering or providing a compound or composition of the invention to a subject by any appropriate means.

The term ‘inhibit’ as used herein in connection with a coronavirus refers to a decrease in binding of a protein encoded by a coronavirus to a cellular target, a decrease in disease symptoms or severity, or a decrease in viral load.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

### DESCRIPTION OF EMBODIMENTS

The present inventors have developed a formulation comprising, thimerosal as an active to inhibit SARS-CoV-2. In particular, the formulation can therefore be used to treat, reduce or prevent coronavirus infection. In some embodiments the formulations additionally comprise one or more of tinocordofolin, cordifolioside A, cordioside, berberine, syringin, tinocordioside, jateroine, jatrorrhizine, magnoflorine, 20-hydroxy ecdysone, palmatine, and piperine.

The formulation is not a vaccine.

Accordingly there is provided the use of a formulation comprising thimerosal as an active to inhibit a coronavirus in a subject. The formulation can be used to treat and/or prevent a coronavirus infection, this may entail one or more of the following:
i) reducing viral load in a subject infected with a coronavirus including but not limited to SARS, MERS, SARS-CoV-2, and in particular, a subject infected with any isolate or strain of SARS-CoV-2.
ii) reducing or ameliorating the risk to a subject of infection with a coronavirus including but not limited to SARS, MERS, SARS-CoV-2, and in particular, the risk of infection with any isolate or strain of SARS-CoV-2.
iii) alleviating symptoms of a coronavirus infection in a subject, having or suspected of having a coronavirus infection including but not limited to SARS, MERS, SARS-CoV-2, and in particular, an infection by any isolate or strain of SARS-CoV-2.
iv) reducing or ameliorating the risk of ADE coronavirus infection in a subject having had or suspected of having had a previous coronavirus infection.

Alternatively, the patient may also be receiving other treatments, such that the formulation is administered in an adjunct therapy. Additional therapies may be those that treat the accompanying symptoms including, without limitation, pain relief such as aspirin, acetaminophen, codeine, and nonsteroidal anti-inflammatory drugs (NSAIDS). In this regard, there is provided a method of treating a coronavirus infection, or a method of reducing symptom severity in a patient having or suspected of having a coronavirus infection, comprising administering an effective amount of the formulation to a subject in need thereof, together with another therapeutic.

### Formulations

The formulations of the invention comprise as actives thimerosal as an active.

Thimerosal, (Sodium; (2-carboxylatophenyl)sulfanyl-ethylmercury) is also known as thiomersal and thimerosol. It is an organomercury compound with an antifungal and antibacterial properties. It is commonly employed as a preservative in vaccine production to avoid fungal or bacterial contamination of multidose preparations, as well as in certain drug products such as ophthalmic, otic, nasal, and topical medicines. Prior to the present invention thimerosal was solely used as a preservative and was not known to have anti-viral properties when administered to a subject as an active.

Thimerosal has the following structure:

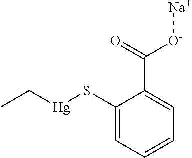

The formulations contain low concentrations of thimerosal in the range of 0.001 to 0.5 mg/100 mL, i.e. 0.00001 mg/mL to about 0.005 mg/mL. For example the concentration of thimerosal in the formulation may be about 0.00001 mg/mL, 0.00005 mg/mL, 0.0001 mg/mL, 0.0002 mg/mL, 0.0003 mg/mL, 0.0004 mg/mL, 0.0005 mg/mL, 0.0006 mg/mL, 0.0007 mg/mL, 0.0008 mg/mL, 0.0009 mg/mL, 0.001 mg/mL, 0.002 mg/mL, 0.003 mg/mL, 0.004 mg/mL, 0.005 mg/mL, 0.006 mg/mL, 0.007 mg/mL, 0.008 mg/mL, 0.009 mg/mL, or 0.01 mg/mL.

In some embodiments the concentration of thimerosal in the formation is about 0.001 mg/mL to about 0.01 mg/mL, for example about 0.004 mg/mL, 0.005 mg/mL, or 0.006 mg/mL, preferably about 0.005 mg/mL.

The formulation may further comprise any one or any combination of additional compounds listed in Table 1.

TABLE 1
Additional components of the formulation

| Compound | |
|---|---|
| class | Compound |
| Alkaloid | Berberine |
| | Choline |
| | Palmatine |
| | Magnoflorine |
| | Tetrahydropalmatine |
| | Tinosporin |
| | Isocolumbin |
| | Jatrorrhizine |
| | N-formlyasimilobine 2-O-β-D−glucopyransyl- |
| | (1→2−β-D-glucopyranoside (tinoscorside A) |
| | N-acetylasimilobine 2-O-β-D-glucopyransyl- |
| | (1→2-β-D-glucopyranoside (tinoscorside B) |
| Glycosides | 18-norclerodane glucoside |
| | Furanoid diterpene glucoside |
| | Tinocordiside |
| | Tinocordifolioside |
| | Cordioside |
| | palmatosides |
| Diterpenoid | Furanolactone |
| lactone | Clerodane derivatives such as |
| | [(5R, 10R)-4R-8R-dihydroxy-2S- |
| | 3R:15, 16-diepoxy-cleroda-13(16), |
| | 14-dieno-17,12S:18,1S-dilactone] |
| | tinosporides |
| Steroids | β-sitosterol |
| | hydroxy ecdysone |
| | ecdysterone |
| | giloinsterol |
| Aliphatic | Octacosanol |
| compounds | Heptacosanol |
| | Nonoacosanol |
| | Nonacosan-15-one dichloromethane |
| Others | 3,(a,4-dihydroxy-3-methoxy-benzyl)-4- |
| | (4-hydroxy-3-methoxy-benzyl)-tetrahydrofuran |
| | N-trans-feruloyl tyramine |
| | Giloin |
| | Tinosporic acid |

In one embodiment the formulation further comprises any one or any combination of additional compounds selected from tinocordofolin, cordifolioside A, cordioside, berberine, syringin, tinocordioside, jateroine, jatrorrhizine, magnoflorine, 20-hydroxy ecdysone, and palmatine. The structures of each of these compounds are presented in Table 2.

TABLE 2
Compound structures

| Compound name | Structure and IUPAC name |
|---|---|
| Tinocordofolin | 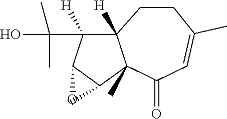 [SMILES: CC1=CC(=O)[C@]2(C)[C@H](C)[C@H](O)[C@@H](C(C)(C)O)[C@@H]2CC1] |
| | (1aS, 1bS,6aS,7S,7aR)-7-(2-hydroxypropan-2-yl)-1b,4-dimethyl- |
| | 1a,5,6,6a,7,7a-hexahydroazuleno [1,2-b]oxiren-2-one |
| Cordifolioside A | 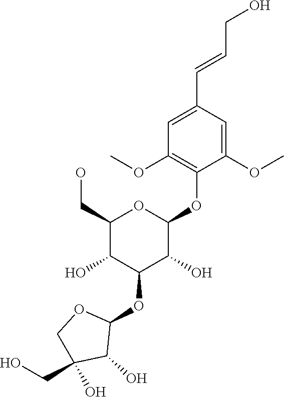 [SMILES: COc1cc(/C=C/CO)cc(OC)c1O[C@@H]1O[C@H](CO)[C@@H](O)[C@H](O[C@@H]2OC[C@](O)(CO)[C@H]2O)[C@H]1O] |
| | 4-[3,4-dihydroxy-4-(hydroxymethyl)oxolan-2-yl]oxy-2- |
| | (hydroxymethyl)-6-[4-(3-hydroxyprop-1-enyl)-2,6- |
| | dimethoxyphenoxyloxane-3,5-diol |
| Cordioside | 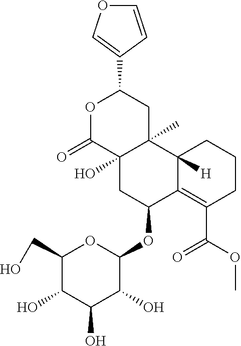 |
| | methyl (2S,4aS,6S, 10aR, 10bS)-2-(furan-3-yl)-4a-hydroxy-10b- |
| | methyl-4-oxo-6-[(2R,3R,4S,5S,6R)-3,4,5-trihydroxy-6- |
| | (hydroxymethyl)oxan-2-yl]oxy-1,2,5,6,8,9,10,10a- |
| | octahydrobenzo [f]isochromene-7-carboxylate |
| Berberine | 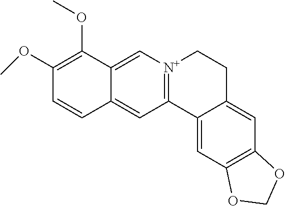 [SMILES: COc1ccc2cc3[n+](cc2c1OC)CCc1cc2c(cc1-3)OCO2] |
| | 16,17-dimethoxy-5,7-dioxa-13- |
| | azoniapentacyclo[11.8.0.02, 10.04,8.015,20]henicosa- |
| | 1(13),2,4(8),9,14,16,18,20-octaene |
| Syringin | 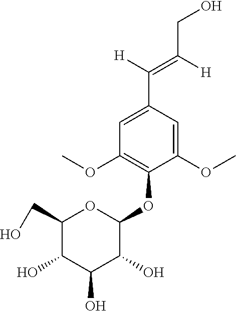 |
| | (2R,3S,4S,5R,6S)-2-(hydroxymethyl)-6-[4-[(E)-3-hydroxyprop-1- |
| | enyl]-2,6-dimethoxyphenoxyloxane-3,4,5-triol |
| Tinocordioside | 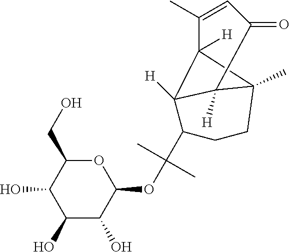 [SMILES: CC1=CC(=O)[C@@H]2C3C(C(C)(C)O[C@@H]4O[C@H](CO)[C@@H](O)[C@H](O)[C@H]4O)CC[C@]2(C)C13] |
| | (1R,2R,8S)-1,5-dimethyl-8-[2-[(2S,3R,4S,5S,6R)-3,4,5- |
| | trihydroxy-6-(hydroxymethyl)oxan-2-ylloxypropan-2- |
| | yl]tricyclo[4.4.0.02,7]dec-4-en-3-one |
| Jateroine | 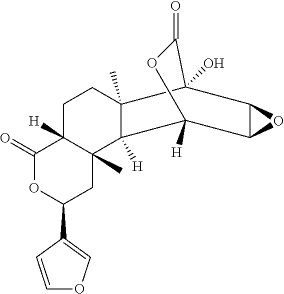 [SMILES: C[C@@]12CC[C@H]3C(=O)O[C@H](c4ccoc4)C[C@@]3(C)[C@@H]1C[C@@H]1COC(=O)[C@@](O)(C[C@H]3O[C@H]3C1)C2] |
| | (1S,2S,3S,5S,8R, 11R, 12S, 13S, 15S)-5-(furan-3-yl)-12-hydroxy- |
| | 3,11-dimethyl-6, 14, 16- |
| | trioxapentacyclo[10.3.2.02, 11.03,8.013, 15]heptadecane-7,17- |
| | dione |
| Jatrorrhizine | 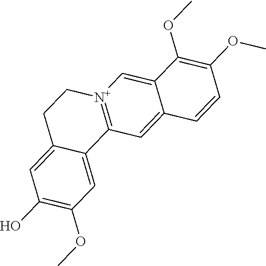 [SMILES: COc1cc2c(cc1O)CC[n+]1cc3c(OC)c(OC)ccc3cc1-2] |
| | 2,9, 10-trimethoxy-5,6-dihydroisoquinolino[2, 1-b]isoquinolin-7- |
| | ium-3-ol |
| Magnoflorine | 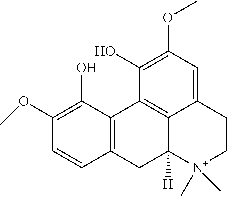 [SMILES: COc1ccc2c(c1O)-c1c(O)c(OC)cc3c1[C@H](C2)[N+](C)(C)CC3] |
| | (6aS)-2, 10-dimethoxy-6,6-dimethyl-5,6,6a,7-tetrahydro-4H- |
| | dibenzo[de,g]quinolin-6-ium-1, 11-diol |
| 20-hydroxy ecdysone | 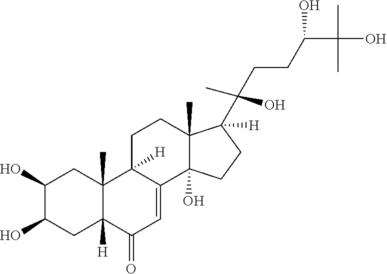 |
| | (2S,3R,5R,9R, 10R, 13R, 14S, 17S)-2,3, 14-trihydroxy-10, 13- |
| | dimethyl-17-[(2R,3R)-2,3,6-trihydroxy-6-methylheptan-2-yl]- |
| | 2,3,4,5,9,11,12,15,16,17-decahydro-1H- |
| | cyclopenta[a]phenanthren-6-one |
| Palmatine | 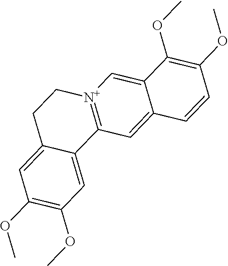 [SMILES: COc1cc2c(cc1OC)-c1cc3ccc(OC)c(OC)c3c[n+]1CC2] |
| | 2,3,9, 10-tetramethoxy-5,6-dihydroisoquinolino[2, 1-b]isoquinolin- |
| | 7-ium |

The combined amount of the additional compounds in the formulation can range from at least 0.1 mg to about 500 mg per 100 mL, i.e from 0.001 to about 5 mg/mL. For example, the amount of the additional compounds in the formulation can be about 0.001 mg/mL, 0.005 mg/mL, 0.01 mg/mL, 0.10 mg/mL, 0.25 mg/mL, 0.50 mg/mL, 0.75 mg/mL, 1.00 mg/mL, 1.25 mg/mL, 1.50 mg/mL, 1.75 mg/mL, 2.00 mg/mL, 2.25 mg/mL, 2.50 mg/mL, 2.75 mg/mL, 3.00 mg/mL, 3.25 mg/mL, 3.50 mg/mL, 3.75 mg/mL, 4.00 mg/mL, 4.25 mg/mL, 4.50 mg/mL, 4.75 mg/mL, or at least 5.00 mg/mL.

In some embodiments the combined amount of the additional compounds in the formulation is 0.10 mg/mL, 0.25 mg/mL, 0.50 mg/mL, 0.75 mg/mL, 1.00 mg/mL, 1.25 mg/mL, 1.50 mg/mL, 1.75 mg/mL, 2.00 mg/mL, 2.25 mg/mL, 2.50 mg/mL, 2.75 mg/mL, 3.00 mg/mL. For example 1.75 mg/mL, 2.00 mg/mL, or 2.25 mg/mL.

The amount of each of the additional compounds in the formulation can be independently selected from a range from at least 0.01 mg to about 50 mg per 100 mL, i.e from 0.0001 to about 0.5 mg/mL. For example, the amount of each of the additional compounds in the formulation can independently be about 0.0001 mg/mL, 0.00025 mg/mL, 0.0005 mg/mL, 0.00075 mg/mL, 0.001 mg/mL, 0.0025 mg/mL, 0.005 mg/mL, 0.0075 mg/mL, 0.01 mg/mL, 0.025 mg/mL, 0.050 mg/mL, 0.075 mg/mL, 0.10 mg/mL, 0.25 mg/mL, or about 0.50 mg/mL.

One example of the source of the additional compounds is an extract from Tinospora cordifolia, for example a stem extract.

In another embodiment the formulation additionally comprises piperine: ((2E,4E)-5-(1,3-benzodioxol-5-yl)-1-piperidin-1-ylpenta-2,4-dien-1-one). The structure of piperine is as follows:

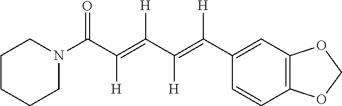 [SMILES: O=C(/C=C/C=C/c1ccc2c(c1)OCO2)N1CCCCC1]

The formulation comprises at least 0.01 mg to about 50 mg of piperine per 100 mL, i.e from 0.001 to about 0.5 mg/mL. For example, the amount of piperine in the formulation can be about 0.001 mg/mL, 0.005 mg/mL, 0.01 mg/mL, 0.050 mg/mL, 0.075 mg/mL, 0.10 mg/mL, 0.125 mg/mL, 0.150 mg/mL, 0.175 mg/mL, 0.200 mg/mL, 0.250 mg/mL, 0.50 mg/mL, 0.75 mg/mL, 0.30 mg/mL, 0.325 mg/mL, 0.350 mg/mL, 0.375 mg/mL, 0.40 mg/mL, 0.425 mg/mL, 0.450 mg/mL, 0.475 mg/mL, or at least 0.50 mg/mL. In some embodiments the amount of piperine in the formulation is 0.10 mg/mL, 0.125 mg/mL, 0.150 mg/mL, 0.175 mg/mL, 0.200 mg/mL, 0.250 mg/mL, 0.50 mg/mL, for example about 0.200 mg/mL, 0.250 mg/mL, or about 0.50 mg/mL.

One source of piperine is a Piper nigrum extract, preferably a seed extract.

In some embodiments the formulation comprises one or more pharmaceutically acceptable excipients including carriers, vehicles and diluents. The term “excipient” herein means any substance, not itself a therapeutic agent, used as a diluent, adjuvant, or vehicle for delivery of a therapeutic agent to a subject or added to a pharmaceutical composition to improve its handling or storage properties or to permit or facilitate formation of a solid dosage form such as a tablet, capsule, or a solution or suspension suitable for oral, parenteral, intradermal, subcutaneous, or topical application. Excipients can include, by way of illustration and not limitation, diluents, disintegrants, binding agents, wetting agents, polymers, lubricants, glidants, stabilizers, and substances added to mask or counteract a disagreeable taste or odor, flavors, dyes, fragrances, and substances added to improve appearance of the composition. Acceptable excipients include (but are not limited to) stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutically acceptable materials. Examples of excipients and their use is described in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

The thimerosal, additional compounds and piperine may be formulated for oral (e.g. sublingual), rectal, nasal, or topical delivery. Preferably the formulation is suitable for sub-lingual administration.

Non-limiting examples of particular formulation types include tablets, capsules, caplets, powders, granules, ampoules, vials, ready-to-use solutions or suspensions, lyophilized materials, creams, lotions, ointments, drops, and suppositories. Solid formlations such as the tablets or capsules may contain any number of suitable pharmaceutically acceptable excipients or carriers described above. The compounds of the invention may also be formulated for sustained delivery.

In one or more preferred embodiments tablets and capsules for oral administration may be in unit dose presentation form, and may contain conventional excipients such as binding agents, for example, acacia, gelatin, sorbitol, tragacanth, or polyvinylpyrollidone; fillers, for example lactose, sugar, maize-starch, calcium phosphate, sorbitol or glycine; tabletting lubricants, for example, magnesium stearate, talc, polyethylene glycol or silica; disintegrants, for example, potato starch; or acceptable wetting agents such as sodium lauryl sulphate. The tablets may be coated according to methods well known in normal pharmaceutical practice.

Oral liquid formulations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example, sorbitol, methyl cellulose, glucose syrup, gelatin, hydroxyethyl cellulose, carboxymethyl cellulose, aluminium stearate gel or hydrogenated edible fats, emulsifying agents, for example, lecithin, sorbitan monooleate, or acacia; non-aqueous vehicles (which may include edible oils), for example, almond oil, oily esters such as glycerin, propylene glycol, or ethyl alcohol; preservatives, for example, methyl or propyl p hydroxybenzoate or sorbic acid; and, if desired, conventional flavouring or colouring agents.

The formulations may comprise fennel oil as a flavouring agent.

In some embodiments the formulation comprises one or more of a humectant, sweetener, preservative, flavouring agent, excipient, diluent, and carrier.

While any humectant known in the art may be used, examples of suitable humectants include one or more of glycerine; propylene glycol; hexylene glycol, butylene glycol; aloe vera; alpha hydroxy acids such as lactic acid; glyceryl triacetate; molasses; quillaia; sodium hexametaphosphate; honey; castor oil; sugar alcohols (sugar polyols) such as glycerol, sorbitol, xylitol, maltitol.

In preferred embodiment the humectant is either or both of glycerine and propylene glycol.

The formulations may be sweetened using concentrated sucrose solution (syrup) or honey (contains fructose). However, artificial sweeteners are preferred. Examples of suitable artificial sweeteners include acesulfame K; alitame; aspartame; dextrose; fructose; galactose; inulin; isomalt; lactitol; maltitol; maltose; mannitol; neohesperidin dihydrochalcone; saccharin; sodium cyclamate; sorbitol; sucralose; sucrose; tagatose; thaumatin; trehalose; and xylitol. Preferably, the artificial sweetener is sucralose.

The formulations will typically include a preservative. Any preservative suitable for ingestion by a human or animal may be used, for example sodium benzoate; benzoic acid; sodium paraben; ethyl paraben; propyl paraben; alcohols; phenols. Preferably the preservative is sodium benzoate.

The formulations will typically include a flavoring agent such as an artificial pineapple flavour. Other suitable flavouring agents include vanillin, ethyl vanillin; menthol; Manzanate (Ethyl 2-methylpentanoate) for an apple flavour; Isoamyl Acetate also known as Isopentyl Acetate for a banana flavour; γ-octalactone for a coconut flavor; cinnamaldehyde for a cinnamon; methyl anthranilate for a grape; carvone & limonene for a caraway flavour; ethyl decadienoate for a pear flavour; ethyl propionate for a fruity flavour; 2,4-Dithiapentane for a truffle flavour; methyl salicylate for a wintergreen flavour; allyl benzoate for a cherry flavour; allyl hexanoate or allyl caproate for a pineapple flavour; anethole, anisaldehyde for an anise flavour; 3,7-Dimethyl-2,6-Octadienal (lemon flavour); d-Limonene, Ethyl Butyrate (orange flavour); a combination of 3-hydroxy-4,5-dimethyl-2(5H)-furanone, ethyl-2-methybutanoate, (E,Z)-2,6-nonadienal, 1-(E,Z)-undecatriene, γ-octalactone with modifiers such as ethyl butanoate for a mango flavour; ketone 1-carvone, limonene, cineole for a spearmint or mint flavour; gingerol, d-camphene and β-phellandrene for a ginger flavour; 3,5-dimethyl-1,2-cyclopentadione for a caramel flavour; linalyl acetate for a lavender flavour; eugenol for a clove flavour; geraniol and I-citronellol for a rose flavour; aldehyde c-14 peach, or gamma-undecanolactone for a peach flavour; raspberry ketone (4-(4-Hydroxyphenyl)butan-2-one) for a raspberry flavour; ethyl methylphenylglycidate for a strawberry flavour; cocoa, cocoa liquor for a chocolate flavour; limonene, cineol for a cardamon flavour; prunasin for a wild cherry flavour; methionine; maltol for a caramel-like odor and taste but in dilute solution, it imparts a sweet, strawberry-like, or pineapple-like flavour and odor; ethyl maltol to provide a caramel-like odor and taste but in a dilute solution, it provides a sweet, fruitlike flavour and odor; ethyl lactate for a sweet, ethereal, fruity, butterscotch buttery smell; monosodium glutamate as a flavour enhancer; citric acid monohydrate as a flavour enhancer for its tart flavour and acidic taste; dibutyl subacute as a flavour adjuvant; adipic acid, also known as hexane dioic acid, for a tart flavour; capric acid for a citrus-like flavour; malic acid to impart a slight apple flavour; thymol/thyme oil for a sharp, almost minty flavour.

In one embodiment the formulation has the composition provided in Table 3.

TABLE 3
Exemplary formulation.

| Component | Quantity | |
|---|---|---|
| Thimerosal (active) | 0.05 | mg |
| Additional compounds: tinocordofolin, | 200 | mg |
| cordifolioside A, cordioside, berberine, | | |
| syringin, tinocordioside, jateroine, | | |
| jatrorrhizine, magnoflorine, 20-hydroxy | | |
| ecdysone, and palmatine (as T. cordifolia | | |
| stem extract) | | |
| Piperine (as P. nigrum seed extract) | 2.5 | mg |
| Sorbitol solution 70% | 19.23 | ml |
| Fennel oil | 0.25 | ml |
| Glycerine (Humectant) | 39.59 | ml |
| Propylene glycol (Humectant) | 4.81 | ml |
| Sucralose (Artificial Sweetener) | 100 | mg |
| Sodium benzoate (Preservative) | 300 | mg |
| Artificial flavour (Pineapple) | 1.5 | ml |
| Purified water | 35 | mL |

The stability of the exemplary formulation has been tested in triplicate over a six month period at room temperature and indicative results are presented in Table 4

TABLE 4
Formulation stability

| Test | | | Results: | Results: | Results: | Results: |
|---|---|---|---|---|---|---|
| parameters | Specifications | | Month 1 | Month 2 | Month 3 | Month 6 |
| Visual | Light brown | | Light brown | Light brown | Light brown | Light brown |
| appearance | colored liquid | | colored liquid | colored liquid | colored liquid | colored liquid |
| | with | | with | with | with | with |
| | characteristic | | characteristic | characteristic | characteristic | characteristic |
| | odor | | odor | odor | odor | odor |
| pH | 5.0 to 6.5 | | 6.1 | 6.1 | 6.1 | 6.1 |
| Assay of bitters | NLT 0.1% | | 0.45% | 0.48% | 0.45% | 0.45% |
| by gravimetry | | | | | | |
| HPLC assay of | NLT 0.001% | | 0.0016% | 0.0018% | 0.0015% | 0.0015% |
| piperine | | | | | | |
| Microbiological Analysis | | | | | | |
| Total aerobic | NMT 1000 | cfu/g | <10 cfu/g | <10 cfu/g | <10 cfu/g | <10 cfu/g |
| count | | | | | | |
| Total yeast and | NMT 100 | cfu/g | absent | absent | absent | absent |

| | | | | | |
|---|---|---|---|---|---|
| moulds | | | | | |
| E. coli | Must be | absent | absent | absent | absent |
| | absent | | | | |
| Salmonella | Must be | absent | absent | absent | absent |
| | absent | | | | |
| Staphylococcus | Must be | absent | absent | absent | absent |
| aureus | absent | | | | |
| Pseudomonas | Must be | absent | absent | absent | absent |
| aeruginosa | absent | | | | |

The formulations are suitable for storge at a temperature between 20 °C to 50 °C. According to stability data, the self-life of the formulation is at least 6 months from the date of manufacturing. For example, the formulation is stable for 1, 2, 3, 4, 5, or at least 6 months at room temperature.

### Methods

The methods and uses described herein comprise the administration of a formulation comprising thimerosal to a subject exposed to a coronavirus, infected by a coronavirus, or a subject suspected of exposure to a coronavirus.

Administration of a formulation comprising thimerosal to a subject inhibits a coronavirus in the subject. The inhibition has a number of beneficial effects such as reducing viral load, preventing or reducing the interaction of the coronavirus with cellular targets. These effects of viral inhibition are useful to prevent or treat (e.g. lessen the severity) of a coronavirus infection.

The formulation can be administered as a precautionary measure to for example healthcare workers or carers or those at risk of exposure to a coronavirus

While infection by one coronavirus serotype or strain may be protective of re-infection by the same serotype or strain, the subject may not be protected by infection with other serotypes or strains. Subsequent infection with a different serotype is thought to result in ADE infection. Accordingly, in some embodiments of the invention, the subject has had a prior coronavirus infection with a different coronavirus serotype or strain. These subjects are at higher risk of ADE coronavirus infection. The methods of the invention are also suitable for treating, preventing or reducing the risk of ADE coronavirus infection.

ADE occurs when circulating antibodies from a previous infection with a different coronavirus serotype lead to increased viral load, caused by cross-reactive but non-neutralizing antibodies. ADE is known to occur with SARS-CoV and MERS-CoV. ADE also results in the increased expression of genes such as those encoding the cytokines interleukin (IL) 8 and tumor necrosis factor (TNF) a which are associated with development of more severe manifestations of an infection.

Accordingly, in a further embodiment of the invention, there is provided a method for preventing or reducing the severity of an ADE coronavirus infection, comprising administering an effective amount of a formulation comprising thimerosal.

There is also provided a use of an effective amount of thimerosal in the preparation of a medicament for preventing ADE coronavirus infection in a subject in need thereof.

In some embodiments administration of the formulation to a subject with a coronavirus infection reduces the amount of detectable coronavirus (cell associated or free virus) by at least 5%. For example the amount of detectable coronavirus may be reduced by at least 5%, at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9%.

The level of detectable coronavirus may be reduced over a period of 1, 2, 3, 4, 5, 6, 7, 8, or more days.

The amount of therapeutically effective formulation that is administered and the dosage regimen for treating a disease condition with the compounds and/or pharmaceutical compositions of the present invention depends on a variety of factors, including the age, weight, sex, and medical condition of the subject, the severity of the disease, the route and frequency of administration, the particular compound employed, as well as the pharmacokinetic properties (e.g., adsorption, distribution, metabolism, excretion) of the individual treated, and thus may vary widely. Such treatments may be administered as often as necessary and for the period of time judged necessary, for example by the subject, their carer or a healthcare provider. One of skill in the art will appreciate that the dosage regime or therapeutically effective amount of the compound to be administrated may need to be optimized for each individual.

The formulation is typically administered sublingually for example via a dropper. Alternatively, the formulations may also be encapsulated and administered via a capsule, tablet, lozenge or the like.

The formulation may be administered over a number of days, for example over a period of 1, 2, 3, 4, 5, 6, 7, 8, 9, 10, 11, 12, 13, 14 or more days.

In one embodiment the formulation is administered at least hourly on the first day, for example on the first day of treatment a dose of the formulation is administered about every 15 minutes for the first hour and then every hour during waking hours.

As treatment progresses the interval between doses may be extended. From the second day of treatment the formulation can be administered two, three, four, five or six or more times.

For example on the second and subsequent days of treatment a dose of the formulation is administered at least three or four times a day, preferably at least six times a day.

In one embodiment on the after the seventh day of treatment a dose of the formulation is administered three times a day.

An exemplary dosage regime is presented in Table 5. Each administration is preferably orally 5 minutes before eating or drinking to avoid dilution.

The volume of an individual dose of the formulation may vary from 0.1 to 1.0 mL depending on the concentration of each constituent in the formulation. In the exemplary formulation provided herein a suitable individual dose is 0.2 to 0.6 mL, preferably 0.4 mL.

TABLE 5
Exemplary dosing regime

| | |
|---|---|
| Schedule | Administration |
| Day 1 | 0.4 ml every 15 minutes for the first hour, |
| | followed by 0.4 ml every hour until bedtime. |
| Days 2 & 3 | 0.4 ml 6 times a day, evenly spaced |
| | during typical waking hours. |
| Days 4 to 7 | 0.4 ml 5 times a day, evenly spaced |
| | during typical waking hours. |
| Day 8 and | 0.4 ml three times a day |
| beyond | |

In another embodiment the formulation may administered one, two, three, four, or more times per day for the treatment period, for example four times a day for 14 days.

The formulations of the present invention may be administered in combination with other agents, for example, other antiviral agents and/or agents suitable for symptomatic relief such as pain killers or expectorants.

The terms ‘combination therapy’ or ‘adjunct therapy’ in defining use of a compound of the present invention and one or more other pharmaceutical agents, are intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations of each agent.

In accordance with various embodiments the formulations may include or be administered in combination with one or more other therapeutic agents. A number of agents are available in commercial use, in clinical evaluation and in pre-clinical development, which could be selected for treatment of coronavirus infection include antiviral agents such as protease inhibitors, helicase inhibitors, entry inhibitors. Other antiviral agents include oseltamivir (Tamiflu®), zanamivir (Relenza®), ribavirin, remdesivir, penciclovir, faviparvir, nafamostat, nitazoxanide, camostat mesylate, interferon α (e.g., interferon α B2), ritonavir, lopinavir, ASC09, azvudine, baloxavir marboxil, darunavir, cobicistat, ivermectin and chloroquine.

Other suitable agents which may be used in combination therapy (for example non-steroidal anti-inflammatories, acetaminophen, codeine, corticosteroids, interferons, etc) will be recognized by those of skill in the art. Suitable agents are listed, for example, in the Merck Index, An Encyclopaedia of Chemicals, Drugs and Biologicals, 12th Ed., 1996, and subsequent editions, the entire contents of which are incorporated herein by reference.

Combination regimens may involve the active agents being administered together, sequentially, or spaced apart as appropriate in each case. Combinations of active agents including compounds of the invention may be synergistic.

The co-administration of the formulation may be effected by the additional therapeutic being in the same unit dose as the formulation, or the formulation and one or more other active agent(s) may be present in individual and discrete unit doses administered at the same, or at a similar time, or at different times according to a dosing regimen or schedule. Sequential administration may be in any order as required, and may require an ongoing physiological effect of the first or initial compound to be current when the second or later compound is administered, especially where a cumulative or synergistic effect is desired.

The co-administration of another therapeutic may be effected by the compounds being in the same unit dose as another active agent, or the compounds and one or more other active agent(s) may be present in individual and discrete unit doses administered at the same, or at a similar time, or at different times according to a dosing regimen or schedule. Sequential administration may be in any order as required, and may require an ongoing physiological effect of the first or initial compound to be current when the second or later compound is administered, especially where a cumulative or synergistic effect is desired.

The methods described herein are useful for reducing the severity one or more symptoms of a corona-virus infection or a symptom of post-acute sequelae of a corona-virus infection, for example reducing a symptom of long-covid.

While acute manifestations of COVID-19 widely affect the pulmonary, cardiovascular, neurological, hematological, and gastrointestinal systems. Its long-term impacts are not well known, but large numbers of COVID-19 survivors present post-acute COVID-19 sequelae. The etiology of post-COVID symptoms is unknown although the most common signs and symptoms reported by COVID survivors include fatigue, cognitive decline, dyspnea, anxiety, weakness, shortness of breath, depression, palpitations, arthralgia, chest and muscle pain which impair their mobility, daily activities, and health-related quality of life (HR-QoL).

Fatigue is one of the most common and severe post-COVID symptoms among 52-58% of COVID-19 survivors and the symptom has been reported to persist for more than a year after the onset of illness. Fatigue is characterized by an ongoing experience of weakness, exhaustion, and increased difficulty doing physical (e.g., walking, climbing stairs, or lifting) or mental work. In post-COVID syndrome (long-covid), fatigue is frequently accompanied by muscle fatigue and fatiguability, which coincides with markers of inflammation and hypoperfusion

Prolonged fatigue after COVID-19 shares clinical manifestations with other post-infectious fatigue syndromes as well as myalgic encephalomyelitis/chronic fatigue syndrome (ME/CFS). In both situations, there are higher levels of circulating proinflammatory cytokines (such as IL-1, IL-4, GM-CSF, IFN-γ, TNF-α, etc.) and CD8+ T cells, which are presumed to be involved in the pathophysiology of these illnesses. In such chronic inflammatory conditions, as well as possibly in log-covid, inflammatory cytokines reduce basal ganglia function and dopamine while increasing neurotransmitter release in the brain, including serotonin, which causes persistent fatigue.

In one embodiment administration of the formulation described herein reduces persistent fatigue in a subject suffering from long-covid, compared to an untreated subject. For example, the persistent fatigue may be characterized by a reduction in a FSSQ (Fatigue Severity Scale Questionnaire) score.

Fatigue can be defined as a subjective experience, and includes such symptoms as rapid inanition, persisting lack of energy, exhaustion, physical and mental tiredness, and apathy. It can be a consequence of many disorders including the post-acute phase of a viral infection and is a noted part of long-covid.

Fatigue can be measured by any method known in the art but one method is the FSSQ which is a commonly and clinically validated fatigue specific 9-item self-reporting questionnaire scale developed.

Other symptoms exhibited by subjects having a coronavirus infection or in the post-acute phase of an infection include inflammation characterized by increased levels of circulating proinflammatory cytokines (such as IL-1, IL-4, GM-CSF, IFN-γ, TNF-α, etc.) and CD8+ T cells. Inflammation in this context may also be characterized by increased levels of chemokines associated with inflammation such as CCL4 (MIP-13)

In one embodiment administration of the formulation described herein reduces inflammation, compared to an untreated subject. This reduction is characterized by a reduction in the levels of one or more circulating proinflammatory cytokines, for example a reduction in IFN-γ and/or CCL4 (MIP-13) levels.

SARS-CoV-2 infection is associated with persistent, relapsing, or new symptoms or other health effects occurring after acute infection, termed post acute sequelae of SARS-CoV-2 infection (PASC), also known as long-covid. There are various methods of characterising PASC, including the PASC Score (Thaweethai et al, JAMA. 2023; 329(22):1934-1946) which involves assessing 12 symptoms with corresponding scores ranging from 1 to 8, where the symptoms are smell/taste, post exertional malaise, chronic cough, brain fog, thirst, palpitations, chest pain, fatigue, sexual desire or capacity, dizziness, gastrointestinal, abnormal movements, and hair loss. A PASC score of 12 or greater is indicative of long-covid. Symptoms (ordered by decreasing frequencies among participants with long-covid) are post exertional malaise, fatigue, brain fog, dizziness, GI symptoms, palpitations, changes in sexual desire or capacity, loss of or change in smell or taste, thirst, chronic cough, chest pain, and abnormal movements. Symptoms correlated with the selected symptoms included dry mouth, weakness, headaches, tremor, muscle and abdominal pain, fever/sweats/chills, and sleep disturbance.

In one embodiment administration of the formulation described herein reduces the PASC Score of a subject compared to an untreated subject.

### Coronavirus

As described herein a formulation comprising thimerosal, one or more of the additional compounds and piperine can be used to inhibit a coronavirus in a subject. In addition, the formulation be used to treat and/or prevent a coronavirus infection.

The methods and uses are applicable to all members of the Coronaviridae (Coronavirus) family.

For example, the coronavirus may be an alphacoronavirus such as coronavirus 229E, coronavirus NL63, porcine epidemic diarrhea coronavirus (PEDV), or swine acute diarrhea syndrome coronavirus (SADS-CoV).

In one embodiment the alphacoronavirus is coronavirus 229E, or coronavirus NL63.

In some embodiments the coronavirus may be a betacoronavirus such as murine coronavirus. In other embodiments the coronavirus is a betacoronavirus 1, for example coronavirus HKU1, murine coronavirus, SARS (severe acute respiratory syndrome)-coronavirus (SARS-CoV), Severe acute respiratory syndrome coronavirus 2 (SARS-CoV-2), MERS (Middle east respiratory syndrome) coronavirus (MERS-CoV), Middle East respiratory syndrome-related coronavirus, or coronavirus OC43.

In some embodiment the coronavirus is coronavirus OC43, SARS-CoV, MERS-CoV or SARS-CoV-2. In one embodiment the coronavirus is SARS-CoV-2.

In some embodiments, reference to SARS-CoV-2 includes reference to any SARS-CoV-2 isolate, SARS-CoV-2 variant, SARS-CoV-2 variant of interest (VOI), or SARS-CoV-2 variant of concern (VOC).

Accordingly, in some embodiments reference to SARS-CoV-2 includes reference to known variants such as the ‘UK variant’, (known as 201/501Y.V1, VOC 202012/01, or B.1.1.7), the ‘South African variant’ (known as 20H/501Y.V2 or B.1.351) and the ‘Brazilian variant’ (known as P.1).

The ‘UK variant’ is characterized by the N501Y mutation in the receptor binding domain (RBD) of the spike protein at position 501. This variant also has several other mutations, including P681H near a furin cleavage site and a 69/70 deletion that likely leads to a conformational change in the spike protein.

The ‘South African variant’ is variant is characterized by multiple mutations in the spike protein, including K417N, E484K, N501Y. This variant does not contain the deletion at 69/70.

The ‘Brazilian variant’ is characterized by three mutations in the spike protein receptor binding domain: K417T, E484K, and N501Y.

Other variants include those with a D614G mutation in the spike protein, and the ‘Cluster 5’ variant, also known as the ‘AFVI-spike’ variant as identified in Denmark which is characterized by mutations including 69-70deltaHV (a deletion of the histidine and valine residues at the 69th and 70th position in the spike protein), Y453F, 1692V, and S1 147L.

The SARS-CoV-2 genome has been sequenced from over 150 isolates. One reference sequence is GenBank NC 045512 (Wang, C. et al. (2020) “The Establishment Of Reference Sequence for SARS-CoV-2 and variation analysis,” J. Med. Virol. 2020 June; 92(6):667-674 (published online 13 Mar. 2020). Comparisons of the sequences of multiple isolates of the virus (MN988668 and NC 045512, isolated from Wuhan, China, and MN938384.1, MN975262.1, MN985325.1, MN988713.1, MN994467.1, MN994468.1, and MN997409.1) reveal greater than 95% identity. Accordingly, the methods described herein are applicable to SARS-CoV-2 variants having at 95%, 96%, 97%, 98%, or 99% sequence identity to a SARS-CoV-2 isolate.

In other embodiments the Coronavirus is a gamma coronavirus such as infectious bronchitis virus, or a delta coronavirus such as bulbul coronavirus HKU11 or porcine coronavirus.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

### Example 1: Shortening Long Covid Symptom Duration

This open label clinical study is designed to evaluate that the exemplary formulation described above shortens Long Covid symptom duration. The study will example 15 patients (male and female) aged from 18 to 99 years over a period of 14 days.

The primary objective is to evaluate the symptom score improvement for Long Covid symptoms after administration of the formulation.

The secondary objective is to assess changes in the biomarkers identified using incelkline kits and other blood parameters and to assess the tolerability of using formulation for a minimum duration of 8 days.

Subjects who meet all of the following criteria will be included in the study:
a) Male and female subjects ranging in age from 18 to 99 years.
b) Willingness to follow the protocol requirements as evidenced by written, informed consent.
c) Mentally, physically and legally eligible to give informed consent
d) Willingness to complete study questionnaires.

Subjects who meet any of the following criteria will be excluded from the study:
a) Patients with pre-existing other severe systemic disease necessitating long-term medication.
b) History of cancer, including solid tumours, hematologic malignancies and carcinoma in situ
c) Any vascular or systemic disorder which could affect any of the efficacy assessments
d) Subjects received other medication (anti-viral) within the 36 hours before entry into the study
e) Evidence of significant uncontrolled co-morbid disease which in the investigator's opinion would jeopardize patient participation.
f) Patients hospitalized or requiring oxygen support.

The primary endpoint is a change from baseline to end of the study in responses to the symptom score and immunological profile among participants consenting to take the formulation. The secondary endpoints are a reduction of biomarker from baseline to end of the study; and/or incidence of adverse events (AEs).

All participants who give consent and are eligible will receive the formulation for a period of 14 days. Subjects will have to visit the clinic on screening visit (visit 1), day −2 (visit 2, randomization visit) which will be 1 to 2 days from the day of screening. Subsequent visits will be on day 8 (visit 3), day 14 (visit 4). All the eligible subjects enrolled into the study shall have to visit the study site on days 1 (baseline visit) and day 14. Physical examination, demographics, vitals, will be recorded on all visits. Day 8 will be a telephonic follow up for symptoms and clinical history for any potential side effects experienced by the participant. A buffer period of ±2 days will be allowed for every visit and beyond which it will be considered as a protocol deviation. Subjective and objective efficacy parameters will be analysed on day 0, day 8 and day 14.

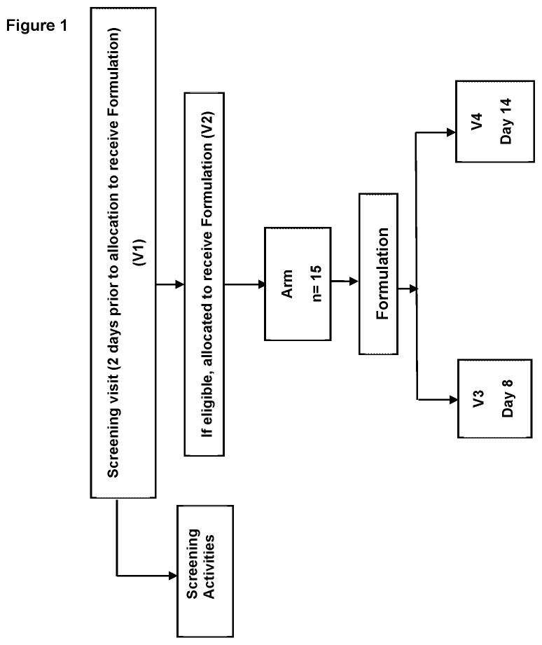

A scheme of the study is provided in FIG. 1. On a Screening Visit (Visit 1 (V1) which is 1-2 days prior to randomization) subjects will be screened for inclusion and exclusion criteria of the study after obtaining the informed consent. Once the subjects are screened and deemed eligible, they will be assigned a unique subject number. Any other symptoms unrelated to study indication will be recorded. Subjects need to come for screening visit in fasting condition. All subjects will be assessed for their physical examinations, vital signs, demographic data; Upon satisfying the inclusion/exclusion criteria eligible subjects will be requested to come for the next visit (V2).

In FIG. 1, the ‘Screening Activities’ are obtaining informed consent; screen subjects by inclusion and exclusion criteria; document the medical history; physical examination; obtain vital signs; obtain heart rate, blood pressure, respiratory rate, and run lab tests.

On the randomization Visit (V2) at day 1 the principal investigator will review all laboratory results before enrolling the subjects into the trial. All subjects will be assessed by the study physician for physical examination, vital signs, medical history, baseline AEs and concomitant medications (if any). All subjects will be assessed for Efficacy Parameters and Biomarkers. Subject will be randomized into study group. Subjects will be given formulation up to Day 14 (V4).

At the Follow up Visit 3 (V3) at 8 days all subjects will be assessed by the study physician telephonically, self-recorded vital signs, relevant medical history, any AEs, concomitant medications and their symptomatic improvement. All subjects will be assessed for Efficacy Parameters. Subjects will be requested to continue taking the formulation up to Day 14 (V4). any reasons for discontinuation will be captured.

At the follow up Visit (V4) at 14 days all subjects will be assessed by the study physician for physical examination, vital signs, medical history, any AEs, concomitant medications and their symptomatic improvement. Haematology, biochemistry will be done. All subjects will be assessed for Efficacy Parameters, Biomarkers.

### Subject Recruitment and Screening

Approximately one site will be chosen for study related activities. Those sites will have adequate facilities for conducting the study. The healthy subjects who are interested will be selected in compliance with the ICH-GCP guidelines.

### Assigning Treatment Groups

Eligible subjects will be allocated to study arm
Study Group (n=15): Formulation

### Treatment Regimen and Administration

Administration: Subjects are advised to take 1 drop (0.4 ml) every 15 minutes for the 1st hour on day 1 and then one drop (0.4 ml) for every hour till 8 hours on day 1 and followed by 1 drop (0.4 ml) every 2 hours for 2 days and later reduced to 1 drop (0.4 ml) 3 times a day (8 hours apart). Keep the solution under the tongue for about 15 seconds before swallowing the liquid in the mouth.

All days the formulation must be administered before any meal. A gap of 10-15 minutes must be observed before taking any fluids or food after taking the drops.

Supply of medication: Subject will be supplied with the Investigational products (IP) at V2 for the duration of the study. Each participant will get 4 bottles of the formulation packed in 2 primary packs containing 2 bottles each. Each bottle has a filler with markings for measuring the 0.4 ml for administration.

### Early Discontinuation

Subjects will be withdrawn from the study if they do not meet inclusion/exclusion criteria.

Withdrawal of subjects from study will be considered, if subject withdraws consent or investigator considers withdrawal in case of non-compliance with protocol violation or loss to follow-up. Withdrawal will also be considered in the interest of the subject due to worsening disease or tolerability issues, such as clinical deterioration or serious AE. The reasons for withdrawal will be recorded in the case report form.

Apart from withdrawals, subjects may also drop out for reasons of non-compliance, lack of efficacy and lack of tolerability and the reasons for drop-out will be recorded in the case report form, dated and signed at the last assessment visit. For all subjects dropping out of the study, efforts will be made to ascertain the reason for drop out.

### Data Collection and Follow-Up for Discontinued Subjects

Even though subjects may be withdrawn prematurely from the study, it is imperative to collect at least safety data on such subjects for the duration of the study. Such data is important to the integrity of the final study analysis since early withdrawal could be related to the safety profile of the study product. If a subject withdraws consent, attempts will be made to obtain at least safety data till the end of study period. Subjects who withdraw from the study due to lack of tolerability will be contacted regularly for safety follow up until the issue is resolved. Subject will be considered as ‘loss to follow up’ after 3 documented attempts to contact (phone calls) are made.

### Number of Subjects

Approximately 20-25 healthy subjects will be screened to select 15 subjects. Those subjects will be allocated to receive the formulation. Any participant who does not consume the product for a minimum of 5 days will be discontinued and not considered for any final efficacy analysis.

Table 6 provides a schedule of events for each subject in the study

TABLE 6
Schedule of events

| | | Visit 2 | | |
|---|---|---|---|---|
| | | (day 1, | | |
| | Screening | Baseline | Visit 3 | Visit 4 |
| | (Visit 1) | visit) | (day 8) | (day 14) |
| | ±/−2 days | ±/−2 days | ±/−2 days | ±/−2 days |
| Inclusion and Exclusion | X | | | |
| Criteria | | | | |
| Informed consent | X | | | |
| Medical history | X | | | |
| Physical examination | X | X | | X |
| Demographics | X | | | |
| Vital Signs | X | X | X | X |
| Haematology | X | | | X |
| Serum Chemistry | X | | | X |
| IP Dispensing and | | X | | X |
| Reconciliation | | | | |
| Post Covid Functional | | X | X | X |
| Status Score | | | | |
| Fatigue Severity Score | | X | X | X |
| Biomarkers | | X | | X |
| Adverse events | | | X | X |
| Concomitant | | X | X | X |
| medications | | | | |

*For all participants-Whole Blood sample for complete hemogram. For participants using the formulation, measurements will be taken of renal function, liver function and inflammation/long covid panel of tests; Interleukins 1, 4, 6, 8 & TNF a and interferon−gamma. In addition, a plasma sample or serum sample for a flow cytometry analysis the presence of S1 protein in non−classical monocytes.

### Medical History and Concomitant Medication

A complete medical history will be recorded during the screening period and review of concomitant medication throughout the study period on all visits. The medical history will be documented on the appropriate section of the CRF. Significant past medical or surgical procedures as well as previous and current co-existent diseases will be included. It also includes relevant medical history for the following body systems: head, neck, eyes, ENT, respiratory, pulmonary, cardiovascular, gastrointestinal, endocrine, hematological, dermatological, renal, neurological, musculoskeletal, psychological/psychiatric etc. Past and present medical history will be noted along with the past and present medications (if any). These will be recorded in the appropriate section of the CRF.

### Physical Examination

A complete physical examination will be conducted at all visits. Height will be measured at screening visit. Physical examination includes the following areas: head, eyes, ears, nose, throat, neck, heart, lungs, abdomen, lymph nodes, extremities, nervous system and skin.

These observations will be recorded appropriately in the CRF.

### Vital Signs and Body Weight

Vital signs (heart rate, blood pressure, oral temperature and respiratory rate after sitting for 5 minutes) and body weight will be recorded at all visits.

### Clinical Laboratory Tests

Each subject will have the clinical laboratory tests listed in table below, performed at screening and at the end of the study. Blood samples will be collected at screening visit, the final visit (V4). Five to eight ml blood will be collected by venipuncture method for clinical laboratory tests at V2, and V4. Clinical laboratory tests include hematology and biochemistry. A total of approximately 15 to 25 ml of blood will be collected from each subject during the course of this study for the laboratory evaluations (Table 7).

TABLE 7
List of Laboratory Tests

| | | | |
|---|---|---|---|
| | | V3 | |
| | | Study | |
| | | period | |
| | V1 | (early | V4 |
| | Screening | discontinuation | Final |
| Parameters | Visit | only) | Visit |

Hematology

| | | |
|---|---|---|
| Hemoglobin | ✓ | ✓ |
| Platelet count | ✓ | ✓ |
| Total leukocyte count | ✓ | ✓ |
| Red blood cells | ✓ | ✓ |
| ESR | ✓ | ✓ |

Differential Count

| | | |
|---|---|---|
| Neutrophils | ✓ | ✓ |
| Lymphocytes | ✓ | ✓ |
| Eosinophil | ✓ | ✓ |
| Monocytes | ✓ | ✓ |
| Basophils | ✓ | ✓ |

Biochemistry

| | | | |
|---|---|---|---|
| Blood glucose | ✓ | | ✓ |
| (Fasting) | | | |
| Serum creatinine | ✓ | | ✓ |
| Blood uric acid | ✓ | | ✓ |
| Blood urea nitrogen | ✓ | | ✓ |
| Bilirubin | ✓ | | ✓ |
| SGOT | ✓ | | ✓ |
| SGPT | ✓ | | ✓ |
| Serum albumin | ✓ | | ✓ |
| Central Biomarker | | | |
| testing incelKINE ™ | | | |
| Molecular testing for | ✓ | ✓ | ✓ |
| Long Covid | | | |

### Adverse Events and Serious Adverse Events

The investigator is responsible for the detection and documentation of events meeting the criteria and definition of an AE, adverse drug reaction (ADR), or serious adverse event (SAE) as provided in this protocol. All AE's and SAE's will be recorded in the source documents and CRF. All AE's and SAE's that occur from the time written informed consent is obtained until completion of the final visit will be entered on the appropriate CRF page. Information to be collected includes the nature, date and time of onset, intensity, duration, causality, action taken and outcome of the event. Even if the AE is assessed by the investigator as not reasonably attributable to the intervention, its occurrence must be recorded in the source documents and reported on the CRF. Details of any medications given to the subject to abate the AE should be recorded on the concomitant medication page.

During the study, the investigator should enquire periodically about the occurrence of AEs and simultaneously address any self-reported adverse events (AE's). The following are examples of open-ended questions that may be used to obtain this information:
“How are you feeling?”
“Have you had any medical problems since your last visit/assessment?”
“Have you taken any new medication, other than those given to you in this study, since your last visit/assessment?”

### Definition of an Adverse Event

An Adverse Event (AE) is any untoward medical occurrence in a subject or clinical investigation of a subject who administered a study product that need not necessarily have a causal relationship with this supplement.

An AE can therefore be any unfavourable and unintended sign (for example, an abnormal product, whether or not considered related to the medicinal product.

Examples of AEs include:
Exacerbation of a pre existing illness following the start of the study
Increase in frequency or intensity of a pre existing episodic event or condition
Condition detected or diagnosed after study product administration even though it may have been present prior to the start of the study

### Adverse Drug Reaction (ADR)

All noxious and unintended responses to a medicinal product related to any dose should be considered adverse drug reactions.

### Definition of a Serious Adverse Event

A SAE is any untoward medical occurrence that at any dose:
Results in death
Is life threatening
Requires inpatient hospitalization or prolongation of existing hospitalization
Results in persistent or significant disability/incapacity
Is a congenital anomaly/birth defect

### Example 2: Case Study 1

A 53 year old male subject tested positive for Covid and experienced body aches, shortness of breath, headache and cough. His primary physician began conventional treatment for a period of three days, this treatment was ineffective with the Covid infection becoming more severe as the subject's oxygen levels were dropping, and breathing was becoming laboured to the point where hospitalisation was indicated. The patient was administered the exemplary formulation described above for a period of two days at which point his symptoms largely resolved (laboured breathing, headache, body aches and poor oxygen saturation). Administration of the formulation was continued for the several more days until all symptoms had fully resolved.

### Example 3: Case Study 2

A 73 year old female patient tested positive for Covid and with a high fever (102 °F/38.9 °C), body aches, chills, extreme fatigue, shortness of breath, and severe headache for approximately five days after which the symptoms gradually resolved. However the patient reported tinnitus in her right ear, lack of focus (‘brain fog’), continued fatigues, and gastrointestinal distress all of which had not been present before the acute phase of her Covid infection. These (long-covid) symptoms continued for several months.

The patient was administered the exemplary formulation described above for a period of 14 days and experienced a complete resolution of all long-covid symptoms except for tinnitus.

### Example 4: Case Study 3

A middle aged male subject tested positive for Covid and experienced body aches, shortness of breath, headache and cough. Conventional treatment for a period of three days was ineffective with the Covid infection becoming more severe and breathing was becoming laboured to the point where hospitalisation was indicated. The patient was administered a formulation comprising thimerosal at a concentration of 0.05 mg/100 mL (without the additional compounds or piperine described above) for a period of 5 days at which point his symptoms largely resolved (laboured breathing, headache, body aches and fever).

### Example 5: Single-Arm, Non-Randomized, Open-Label Prospective Study

A single-arm, non-randomized, open-label prospective study on the exemplary formulation described in Table 3 (the Formulation) was conducted. The efficacy and safety of the formulation was evaluated as adjunctive therapy for a maximum of 14 days for COVID-19 survivors with persistent COVID-19 symptoms. The study was carried out in accordance with the Declaration of Helsinki, relevant ethical guidelines for biomedical research, and ICH guidelines for good clinical practice (GCP).

All potential participants were voluntarily recruited, and informed consent was obtained prior to the initiation of any study-related procedures.

Male and female patients over the age of 18 who had been diagnosed with COVID-19 by RT-PCR and who had persistent mild to moderate post-COVID symptoms lasting 4 to 12 weeks since disease onset were recruited. Patients were excluded if they were asymptomatic, had severe systemic diseases, uncontrolled comorbid conditions, vascular disorders, cancers, were hospitalized or required oxygen support, or had received antiviral medication in the last 36 hours. Potential participants were screened for eligibility, and demographic data was collected.

Subjects were instructed to take 0.4 mL of the Formulation 4-6 times daily, 5 minutes before eating or drinking. After 14 days of treatment, the efficacy and safety were evaluated.

The primary outcomes were improvements in chronic fatigue and other symptoms associated with long COVID patients as measured by Fatigue Severity Scale Questionnaires (FSSQ) (Valko et al. Validation of the fatigue severity scale in a Swiss cohort. Sleep. 2008; 31:1601-7.) and a modified version of the COVID-19 Yorkshire Rehabilitation Screening (C19-YRS) checklist (Sivan et al. Assessing long-term rehabilitation needs in COVID-19 survivors using a telephone screening tool (C19-YRS tool). Adv Clin Neurosci Rehabil. 2020; 19:14-7).

Fatigue severity was determined using self-administered FSSQ questionnaires (9 items). Each item was scored on a 7-point scale, with 1 representing strongly disagree and 7 representing strongly agree. Higher FSS scores for each item indicate more severe fatigue, and if an FSSQ score≥4 indicates clinically significant fatigue. The final score was the average of the 9 items. Prolonged COVID symptoms were assessed using a modified C19-YRS tool consisting of 18 items rated on an 11-point rating scale as 3=mild symptom, 3-5=moderate symptom, and 6-10=severe symptom. The C19-YRS is well validated tool that correlates well with exercise tolerance, lower limb strength and mobility, anxiety, depression and HR-QoL. The C19-YRS checklist analysed symptoms related to body functions and structures (12 items), activity limitations (5 items), and personal factors (1 item). After 14 days of treatment, the FSSQ and C19-YRS scores were evaluated.

Secondary outcomes included changes in plasma levels of inflammatory cytokines (IL-2 and IFN-γ) and chemokines (CCL4 (MIP-1β). IL-2, IFN-γ, and CCL4 (MIP-1β) levels in plasma were measured using multiplex bead-based flow cytometry (IncellKINE, IncellDx, Inc.). The assay was carried out in a 96-well microplate, and samples were analysed using a CytoFlex LX 3-laser flow cytometer (Beckman Coulter Life Sciences, USA) and Kaluza Analysis Software (Beckman-Coulter, Miami, FL). Changes in laboratory parameters, such as the complete blood count (CBC), ESR, renal function, and liver function parameters from baseline, were evaluated as part of the safety study.

A per-protocol (PP) population analysis was used to assess efficacy. Demographic data were presented as mean, percentage, and standard deviation (SD). Efficacy parameters, such as FSSQ and C19-YRS scores, were compared between baseline data and final follow-up data using the Wilcoxon signed-rank test. To compare the mean change in plasma IL-2, IFN-γ, and CCL4 (MIP-13) levels from baseline to the final visit, the Wilcoxon signed-rank test was used. Unless otherwise stated, all hypotheses were tested at a significance level of 0.05. All statistical analysis was carried out using SSPS software version 10.0.

A total of 15 participants were included in the study, of whom 9 (60%) were men and 6 (40%) were women. The subjects' average age was 47.0±10.58 years. The mean height, weight, and BMI of the participants were 161±8.16 cm, 61.60±7.49 kg, and 23.71±1.17 kg/m², respectively.

The fatigue severity in COVID-19 survivors was assessed using the FSSQ on days 1 and 14. The results revealed that patients consistently had higher FSSQ scores (i.e., >4) for each item as well as a higher mean FSSQ score at day 1. The FSSQ score of each item was significantly reduced after 14 days of treatment, and the changes were statistically significant (Table 8). The mean FSSQ score also decreased significantly (p<0.001) from day 1 (43.30±7.77) to day 14 (32.30±5.35)

TABLE 8
FSSQ scores after 14 days of treatment

| | Day 1, | Day 14, | | |
|---|---|---|---|---|
| | N = 15 | N = 15 | | |
| | FSSQ score | FSSQ score | | |
| FSSQ | (mean ± SD) | (mean ± SD) | Z score | P-value |
| Motivation lower when fatigued | 4.80 ± 1. | 4.00 ± 0.76 | −2.164 | 0.030 * |
| Exercise brings on my fatigue | 5.00 ± 1.07 | 3.93 ± 0.83 | −2.739 | 0.006 ** |
| I am easily fatigued | 4.67 ± 1.35 | 3.80 ± 0.86 | −2.124 | 0.034 * |
| Fatigue interferes with my physical | 4.80 ± 1.01 | 3.73 ± 0.80 | −3.066 | 0.002 ** |
| functioning | | | | |
| Fatigue causes frequent problems | 4.80 ± 0.78 | 3.33 ± 0.82 | −3.115 | 0.002 ** |
| for me | | | | |
| My fatigue prevents sustained | 4.87 ± 0.92 | 3.60 ± 0.83 | −2.994 | 0.003 ** |
| physical functioning | | | | |
| Fatigue interferes with carrying | 4.93 ± 0.96 | 3.67 ± 0.98 | −3.079 | 0.002 ** |
| certain out certain duties & | | | | |
| responsibilities | | | | |
| Fatigue is among my 3 most | 4.60 ± 0.83 | 3.27 ± 0.88 | −3.115 | 0.002 ** |
| disabling symptoms | | | | |
| Fatigue interferes with work, | 4.87 ± 0.92 | 3.27 ± 1.03 | −3.247 | 0.001 *** |
| family & social life | | | | |
| Total fatigue scores | 43.30 ± 7.77 | 32.30 ± 5. | 35 −3.295 | 0.001 *** |

Significance levels * p < 0.05, ** p < 0.01 and *** p < 0.001.

### Assessment of Long COVID Symptoms by C19-YRS Checklist

The severity of long COVID symptoms was assessed by the C19-YRS checklist at days 1 and 14. Among the Body Functions and Structures items, the intensity of the breathlessness on climbing stairs, fatigue, continence, cognition, anxiety, and depression at day 1 were the prominent symptoms compared to other symptoms, with C19-YRS reporting intensities of 5.43±1.22, 6.08±1.04, 6.0±0.78, 5.17±1.27, 6.00±0.78, 5.40±0.91 and 5.27±1.10, respectively (Table 9). After 14 days of treatment, all these symptoms were present but significantly reduced. Improvements in fatigue, continence, cognition, anxiety, and depression symptoms were found to be statistically significant. Among the activities, a statistically significant improvement in mobility, personal care, usual activities, and social role were observed after 14 days of treatment compared to day 1. The global health perception was also statistically significantly reduced from 3.47±1.51 (day 1) to 2.33±1.50 (day 14).

TABLE 9
Intensity change in long COVID symptoms assessed by C19-YRS.

| | | | | |
|---|---|---|---|---|
| | Day 1, | Day 14, | | |
| | N = 15 | N = 15 | | |
| | Score | Score | | |
| C19-YRS items | (mean ± SD) | (mean ± SD) | Z score | P-value |

Body Functions and Structures

| | | | | |
|---|---|---|---|---|
| Breathlessness at rest | 4.13 ± 1.96 | 4.27 ± 2.02 | −0.816 | 0.414 |
| Breathlessness dressing | 2.80 ± 1.01 | 2.87 ± 1.06 | −1.0 | 0.317 |
| Breathlessness stairs | 5.43 ± 1.22 | 5.25 ± 1.67 | −1.0 | 0.317 |
| Cough/throat | 3.40 ± 1.34 | 3.33 ± 0.58 | −0.447 | 0.655 |
| sensitivity/voice | | | | |
| Swallowing | 2.50 ± 0.71 | 2.80 ± 2.39 | na | na |
| Fatigue | 6.08 ± 1.04 | 4.85 ± 1.41 | −2.858 | 0.004 ** |
| Continence | 6.00 ± 0.78 | 4.43 ± 1.22 | −3.236 | 0.001 *** |
| Cognition | 5.17 ± 1.27 | 4.25 ± 1.71 | −2.373 | 0.018 * |
| Pain/discomfort | 1.50 ± 0.58 | 2.50 ± 1.69 | −0.447 | 0.655 |
| Anxiety | 5.40 ± 0.91 | 3.87 ± 1.55 * | −3.108 | 0.002 * |
| Depression | 5.27 ± 1.10 | 3.29 ± 1.64 | −3.108 | 0.002 ** |
| PTSD screen | 1.50 ± 0.71 | 0.50 ± 0.71 | −1.0 | 0.317 |

Activities

| | | | | |
|---|---|---|---|---|
| Communication | 1.80 ± 1.30 | 0.75 ± 0.96 | −1.414 | 0.157 |
| Mobility | 5.60 ± 1.50 | 3.87 ± 1.64 | −3.354 | 0.001 *** |
| Personal care | 5.14 ± 1.99 | 3.38 ± 2.06 | −2.831 | 0.005 |
| Usual activities | 5.20 ± 1.78 | 3.60 ± 1.72 | −3.105 | 0.002 * |
| Social role | 5.13 ± 1.46 | 2.80 ± 1.70 | −3.471 | 0.001 *** |

Personal factors

| | | | | |
|---|---|---|---|---|
| Global health | 3.47 ± 1.51 | 2.33 ± 1.50 | −2.358 | 0.018 ** |

Significance levels * p < 0.05, ** p < 0.01 and *** p < 0.001.

Table 10 summarizes the plasma levels of IL-2, IFN-γ, and CCL4 (MIP-1β). Plasma levels of IL-2, IFN-γ, and CCL4 (MIP-1β) were measured on days 1 and 14. Table 3 shows that IL-2 levels increased slightly from 4.74±2.45 (day 1) to 5.01±2.90 (day 14), whereas IFN-γ and CCL4 (MIP-1β) levels decreased from 37.58±86.27 to 18.23±22.61 and 1527.07±2185.50 to 1883.81±3834.16, respectively. However, the changes were not statistically significant. We calculated the post-acute sequelae of COVID-19 (PASO) disease score based on the findings using the Patterson et al. [21]proposed formula: S1=(IFN-γ+IL-2)/CCL4 (MIP-1β) with optimized threshold for S1=0.5. At day 1, all patients had a higher PASO score (>0.5), and after 14 days of treatment, there was a trend in the reduction of PASO score among the 11 participants, but 4 patients showed a reversal of the trend in PASO score (Table 11).

TABLE 10
Plasma levels of cytokines/chemokines before and after treatment

| | Day 1, N = 15 | Day 14, N = 15 | |
|---|---|---|---|
| Cytokine/ | Concentration | Concentration | |
| Chemokine | (mean ± SD) | (mean ± SD) | P-value |
| IL-2 | 4.74 ± 2.45 | 5.01 ± 2.90 | 0.865 |
| IFN-Y | 37.58 ± 86.27 | 18.23 ± 22.61 | 0.191 |
| CCL4 (MIP-1B) | 1883.81 ± 3834.16 | 1527.07 ± 2185.50 | 0.955 |

TABLE 11
PASC Score among the participants before and after treatment

| | PASC Score | PASC Score | |
|---|---|---|---|
| Patient | (Day 1) | (Day 1) | Score diff. |
| P01 | 8.80 | 2.79 | −6.01 |
| P02 | 7.40 | 9.31 | 1.91 |
| P03 | 4.31 | 2.77 | −1.54 |
| P04 | 3.82 | 23.63 | 19.81 |
| P05 | 345.85 | 72.36 | −273.49 |
| P06 | 2.69 | 2.04 | −0.66 |
| P07 | 11.22 | 3.83 | −7.38 |
| P08 | 25.95 | 3.16 | −22.79 |
| P09 | 11.24 | 48.82 | 37.59 |
| P10 | 23.94 | 17.77 | −6.17 |
| P11 | 19.50 | 11.09 | −8.41 |
| P12 | 50.38 | 4.32 | −46.06 |
| P13 | 3.03 | 55.90 | 52.87 |
| P14 | 29.46 | 2.32 | −27.14 |
| P15 | 18.75 | 16.78 | −1.97 |

### Safety

The safety parameters included CBC, ESR, and liver and kidney function parameters that were evaluated before and after 14 days of treatment. On day 14, analysis of safety parameters showed no significant differences compared to day 1, and all parameters were within the normal range. There were no adverse or serious reactions reported during the treatment period.

### Discussion

Long-term COVID syndrome (the post-acute sequelae of SARS-CoV2 infection) is a growing public health problem. There is increasing evidence that a significant proportion of COVID-19 survivors develop a chronic and debilitating set of multisystem signs and symptoms. It has a negative impact on society, the economy, and patients' quality of life, physical functioning, work participation, care dependency, and activities of daily living. Long COVID has been associated with over 60 physical and psychological symptoms. Profound fatigue is one of the most commonly reported symptoms in post-acute COVID-19 patients, with a prevalence of 52% within 3 weeks of hospital discharge. The persistence of fatigue following an acute COVID infection is also associated with episodes of overwhelming exhaustion, depressive symptoms, poor sleep quality, memory impairment and low energy levels, all of which influence quality of life. There is currently no specific pharmacotherapy available for the treatment of long COVID. Recently, efforts have been made to understand the cause, epidemiological burden, and potential biomarkers of long COVID.

This study aimed to evaluate the efficacy of the exemplary formulation in Table 3 for improving long COVID symptoms, particularly persistent fatigue in COVID-19 survivors. The findings revealed that all patients experienced clinically significant fatigue prior to treatment, as evidenced by a higher FSSQ score (≥4) for each question as well as an overall FSSQ score (>40). After 14 days of treatment, the Formulation significantly reduced FSSQ scores. The C19-YRS test was also used in this study to assess the effect of CelWel on post-COVID symptoms. Results showed that the Formulation significantly improved some symptoms related to the body's functions and structures, including fatigue and limitations in activities. Therefore, Formulation was found to be effective to ameliorate long COVID symptoms, including persistent fatigue in COVID-19 survivors.

Plasma levels of IL-2, IFN-γ, and CCL4 (MIP-1β) were measured to see if the treatment affected patients' immunogenic profiles. While the Formulation had no effect on the proinflammatory cytokine IL-2, it did downregulate IFN-γ and CCL4 (MIP-1β). In addition, after treatment, the PASC score decreased in 11 patients, indicating a reduction of the inflammatory state by preventing the production of proinflammatory cytokines in patients with prolonged COVID-19. However, four patients had the opposite trend in PASC score and would have required a large or very prolonged dose of the Formulation to improve their PASC score.

### ABBREVIATIONS

ALT: alanine transaminase; AST: aspartate aminotransferase; BUN: blood urea nitrogen; C19-YRS: COVID-19 Yorkshire Rehabilitation Screening; CBC: complete blood count; CCL4: chemokine (C-C motif) ligand 4; COVID-19: coronavirus disease 19; EBV: Epstein Barr virus; ESR: erythrocyte sedimentation rate; FSSQ: fatigue severity scale questionnaires; GCP: good clinical practice; HPV: human parvovirus; HR-QoL: health-related quality of life; ICH: International Conference on Harmonization; MERS: middle east respiratory syndrome; MIP-1β: macrophage inflammatory protein-1; PASC: post-acute sequelae of COVID-19; PTSD: posttraumatic stress disorder; RANTES: regulated upon activation, normal T cell expressed and presumably secreted; RBS: random blood sugar; RT-PCR: reverse transcription polymerase chain reaction; SARS: severe acute respiratory syndrome; SARS-CoV-2: severe acute respiratory syndrome coronavirus 2

1-41. (canceled)

42. A formulation comprising thimerosol for inhibiting a corona virus in a subject.

43. The formulation of claim 42 further comprising one or any combination of additional compounds selected from tinocordofolin, cordifolioside A, cordioside, berberine, syringin, tinocordioside, jateroine, jatrorrhizine, magnoflorine, 20-hydroxy ecdysone, palmatine, piperine, and fennel oil.

44. The formulation of claim 42, further comprising one or more of a humectant, sweetener, preservative, flavouring agent, excipient, diluent, and carrier.

45. The formulation of claim 42, comprising, per 100 mL, about 0.05 mg thimerosol, about 200 mg of the additional compounds; about 2.5 mg piperine; about 19.23 mL 70% sorbitol; about 0.25 mL fennel oil; about 39.59 ml glycerine; about 4.81 mL propylene glycol; about 100 mg sucralose; about 300 mg sodium benzoate; and about 1.5 mL artificial pineapple flavour.

46. The formulation of claim 45, wherein the additional compounds are provided by a Tinospora cordifolia stem extract.

47. The formulation of claim 43, wherein the piperine is provided by a Piper nigrum seed extract.

48. The formulation of claim 42, wherein the subject is a human.

49. The formulation of claim 42, wherein the coronavirus is coronavirus 229E, coronavirus NL63, coronavirus OC43, SARS-CoV, MERS-CoV, SARS-CoV-2, or variants thereof.

50. A method of inhibiting a coronavirus in a subject, treating, preventing, or reducing the risk of a coronavirus infection in a subject, or reducing a symptom of a coronavirus infection, the method comprising administering to the subject a therapeutically effective amount of the formulation of claim 42.

51. The method of claim 50, wherein the level of detectable coronavirus in the subject is reduced by at least 5%.

52. The method of claim 50, wherein the level of detectable coronavirus, or a symptom of a coronavirus infection is reduced over a period of 1 or more, 2 or more, 3 or more, 4 or more, 5 or more, 6 or more, 7 or more, 8 or more, 9 or more, 10 or more, 11 or more, 12 or more, 13 or more, or 14 or more days after the first administration of the formulation.

53. The method of claim 50 wherein the formulation is administered orally or sublingually.

54. The method of claim 53 wherein the formulation is administered over a period of 1 or more, 2 or more, 3 or more, 4 or more, 5 or more, 6 or more, 7 or more, 8 or more, 9 or more, 10 or more, 11 or more, 12 or more, 13 or more, or 14 or more days.

55. The method of claim 54, wherein a dose of the formulation is administered at least hourly on the first day.

56. The method of claim 54, wherein:
on the first day of treatment a dose of the formulation is administered about every 15 minutes for the first hour and then every hour during waking hours;
on the second and third days of treatment a dose of the formulation is administered four or more times a day; and/or
on the fourth and subsequent days of treatment a dose of the formulation is administered three, four, or six times a day.

57. The method of claim 56, wherein:
on the fourth to seventh days of treatment a dose of the formulation is administered four times a day; and/or
after the seventh day of treatment a dose of the formulation is administered three times a day.

58. The method of claim 54, wherein the formulation is administered four times a day.

59. The method of claim 50, wherein the subject is a human.

60. The method of claim 50, wherein the coronavirus is coronavirus 229E, coronavirus NL63, coronavirus OC43, SARS-CoV, MERS-CoV, or SARS-CoV-2.

61. The method of claim 50, wherein:
the symptom of long-covid is persistent fatigue or inflammation; the reduction in persistent fatigue is characterized by a reduction in a FSSQ (Fatigue Severity Scale Questionnaire) score;
the reduction in inflammation is characterized by a reduction in IFN-γ and/or CCL4 (MIP-1p) levels; or
the reduction of a symptom is characterized by a decreased post-acute sequelae score of COVID-19 (PASC) Score.
